(19) United States
(12) Patent Application Publication
(10) Pub. No.: US 2002/0068055 A1
(43) Pub. Date: Jun. 6, 2002

(54) Preventive and therapeutic antiviral drug for aids, hepatitis B, hepatitis C and influenza

(76) Inventors: Shigemi Fujisaki, Nishinomiya-shi (JP); Mitsuo Honda, Mitaka-shi (JP)

Correspondence Address: NIXON PEABODY, LLP, 8180 GREENSBORO DRIVE, SUITE 800, MCLEAN, VA, 22102, US

(21) Appl. No.: 09/932,966

(22) Filed: Aug. 21, 2001

(30) Foreign Application Priority Data
Sep. 18, 2000 (JP) 2000-282462

(51) Int. Cl. A61K 38/46

(52) U.S. Cl. 424/94.67

(57) ABSTRACT

The present invention provides a preventive and therapeutic antiviral drug for AIDS, hepatitis B, hepatitis c and influenza containing MEP-F (metalloendopeptidase-F) and proteases (protein decomposition enzymes) as effective ingredients.

Viruses should pass through CD4 receptors on the surface of the cell membrane for invading into the cell. However, since MEP-F extinguishes the activity of CD4 to prevent the viruses from invading into the cell, growth and proliferation of the virus are inhibited, manifesting preventive and therapeutic efficacy for viral infection.

### BACKGROUND OF THE INVENTION

1. Field of the Invention

The present invention relates to a preventive and therapeutic antiviral drug for AIDS, hepatitis B, hepatitis C and influenza.

2. Description of the Related Art

Viruses take advantage of cell side receptors such as CD4 and chemokine for invading in the cells, and produce DNAs with reverse transcriptases using RNAs as templates. The DNAs are translocated in the nucleus and stay there as proviruses by being fused with genes in the nucleus.

When the cell is stimulated in various manner, proteins are produced based on the provirus, and the proteins are modified into structural proteins for constructing viral particles by the action of proteases. Two pairs of the concomitantly produced RNA genes and reverse transcriptases are translocated in the cell membrane, form virions utilizing native lipid bilayers of the cell membrane, and are discharged out of the cells by budding.

Reverse transcriptase inhibitors such as AZT, which inhibit reverse transcription from RNA to DNA after invasion of the virus, have been already developed. Recently, protease inhibitors have been developed, and are used for treating HIV infected patients in combination with the reverse transcriptase inhibitor. The protease inhibitor inhibits the action of protease that cleaves protein materials manufactured by transcription and translation from provirus DNAs and processes the cleaved proteins as structural proteins of the virus.

Although efficacy of these two kinds of inhibitors have been elucidated, only a limited number of infected patients take these drugs because of the issues relating to drug tolerance, adherence to complicated dosing regimens, and high medication expenditures. In addition, development of antiviral drugs of the next generation is a great problem in medication of viral infections considering the efficacy of existing antiviral drugs.

Safety and regimen of MEP-F we have developed have been confirmed from clinical performances as anti-inflammatory drugs, and the drag is expected to be a therapeutic agent with low medication expenditures. The action elucidated in this invention includes antiviral effects (including anti-HIV effect) of MEP-F (metalloendopeptidase F) and an ability for effectively decomposing the cell side CD4 receptor. In other words, it is an object of the present invention to reduce or lower the incidence of invasion of viruses into the cell by selectively cleaving the CD4 molecule.

### SUMMARY OF THE INVENTION

While the mechanism for preventing the virus from proliferating after being discharged in the blood has been investigated with respect to growth and proliferation of the virus, the main objects thereof have been inhibition of protease that inhibits proteins from being decomposed, and inhibition of the reverse transcriptase.

We have elucidated the antiviral effect of MEP-F (metalloendopeptidase-F). In addition, we have succeeded in extinguishing the activity of CD4 using MEP-F and other proteases for preventing the virus from invading in the cell.

The therapeutic and preventive antiviral drug according to the present invention contains MEP-F and other proteases as effective ingredients, and their efficacy is applicable to AIDS, hepatitis B, hepatitis C and influenza.

### BRIEF DESCRIPTION OF THE DRAWINGS

FIG. 1 is a diagram showing that CD4 gradually decreases at 15, 30, 60 and 120 minutes after administration of MEP-F from an initial concentration of 10 μg/ml;

FIG. 2A is a distribution diagram showing the relation between the CD4 and CD62L expression revels after administration of 10 μg/ml of MEP-F, and indicates that CD4 remains at a 10² expression level on the cell;

FIG. 2B is a distribution diagram showing the relation between the CD4 and CD62L expression revels after administration of 100 μg/ml of MEP-F, and indicates that CD62L remains while CD4 has been extinguished;

FIG. 3 is a diagram showing the relation between the administration level of MEP-F and CD62L expression level, and indicate that CD62L is so insensitive to MEP-F that it is not extinguished at an administration level of MEP-F of 100 μg/ml;

FIG. 4 illustrates additional proliferation of the virus discharged from the cell after proliferation in the cell; and

FIG. 5 illustrates how MEP-F protects the cell from being invaded with the virus.

### DESCRIPTION OF THE PREFERRED EMBODIMENTS

MEP-F (metalloendopeptidase-F) as an effective ingredient according to the present invention is a neutral protein decomposition enzyme that extinguishes the CD-4 activity, and has the following chemical and physical properties:

(1) a molecular weight of 43,000±5,000 as determined by SDS electrophoresis, and a molecular weight of 28,000±7,000 as determined by gel filtration using TSK 3000 SW;

(2) an isoelectric point pI of 4.76±1.0;

(3) substrate specificity against casein, -X-Leu-Y- or Cbz-Cly-Leu-NH₂, and X-Phe-Y- or Cbz-Gly-Phe-NH₂ for cleaving at X-Phe or Gly-Lue bond, and at X-Phe or Gly-Phe bond;

(4) an optimum pH range of 5.5 to 9.5, or 6 to 8, for enzyme activity;

(5) a stable pH range of 4 to 10, or 6 to 8, for enzyme activity;

(6) inhibitors such as EDTA, phosphoramidone and leucine

(7) solubility in water and insolubility in acetone and ethanol; and

(8) a Rf value of 0.08 in 7.0% polyacrylamide gel electrophoresis.

The protease as an effective ingredient according to the present invention comprises, for example, trypsin, α-chymotrypsin, bromelain, papain, Serratia protease, peptidase, sepahrase, pronase, prozyme, urokinase, pancreatin, fibrinolysin, elecodase, collagenase, gelatinase and matrix metalloprotease, and either one or an appropriate combination of at least two of them are used.

a) Observation of the Effect of MEP-F when Various Combinations of HIV (AIDS Virus) and MEP-F were Allowed to React with Normal Cells

i) Growth and proliferation of HIV were completely suppressed by adding MEP-F after cultivating the cells for 24 hours by adding HIV.

ii) Growth and proliferation of HIV were completely suppressed after cultivating the cells for 24 hours by adding HIV and MEP-F.

iii) Growth and proliferation of HIV were completely suppressed by adding HIV and MEP after cultivating the cell for 24 hours.

It was made clear from the observations above that MEP-F suppresses growth and proliferation of HIV.

b) Suppression of HIV from Invading in the Cell by the Action of MEP-F:

Helper cells have two kinds of receptors of CD4 and CD62L, and have activities for receiving HIV.

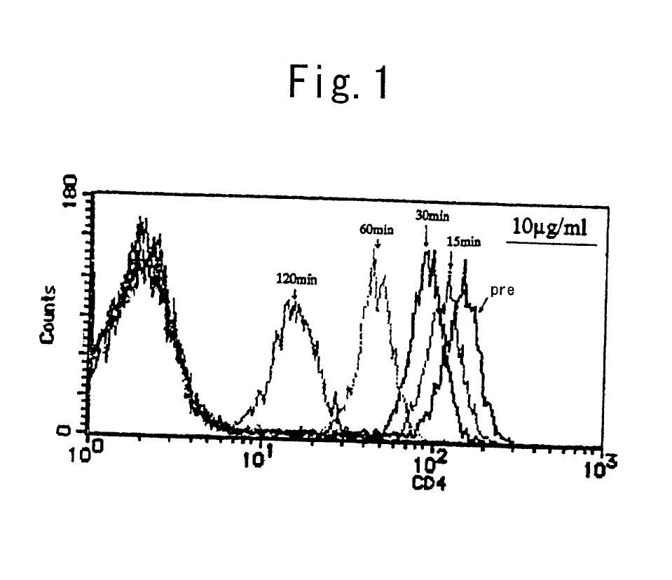

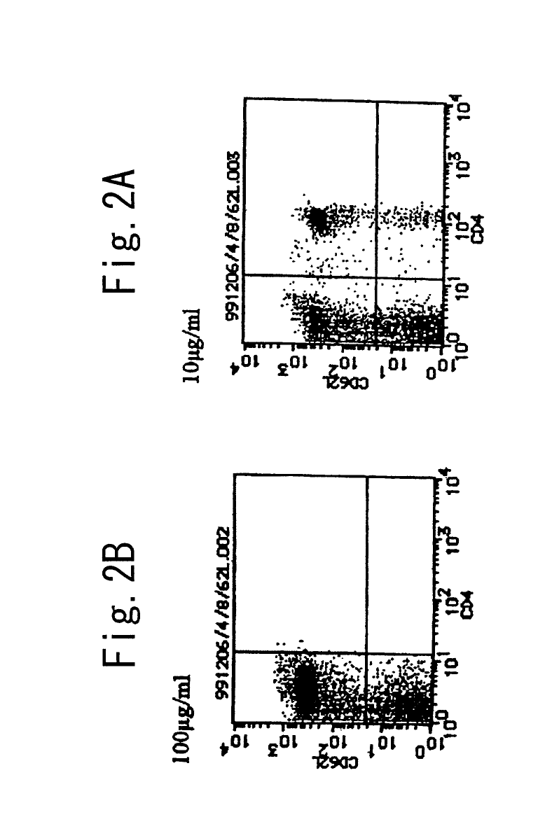

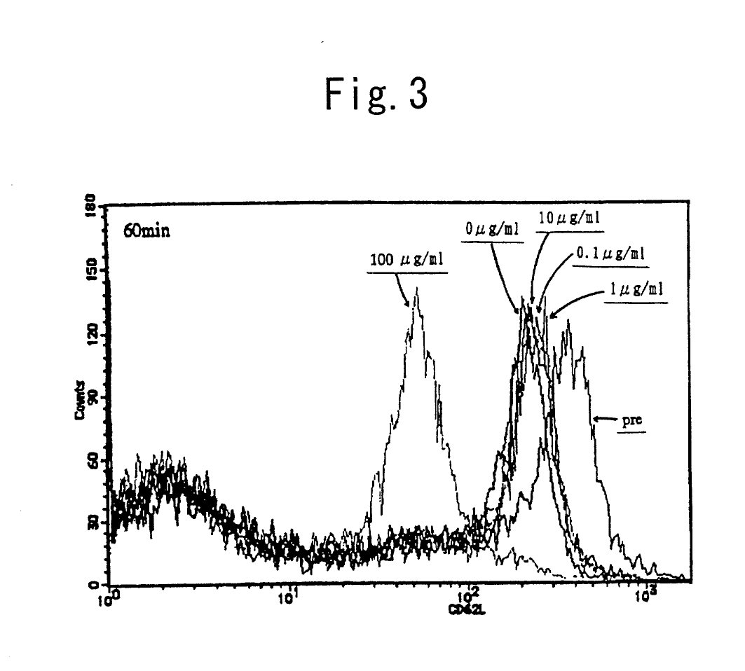

As shown in FIG. 1, administration of MEP-F in a concentration of 10 μg/ml permits the number of CD4 to gradually decrease with time as compared with the number before administration. The cells with a CD4 expression level of 10² are left behind as shown in FIG. 2A when the administration level of MEP-F is 10 μg/ml. However, the CD-4 activity is completely extinguished at a MEP-F administration level of 100 μg/ml as shown in FIG. 2B, while the activity of CD62L is little affected by administration of MEP-F. FIG. 3 shows that CD62L is hardly affected by MEP-F, and remains not extinguished at an administration level of MEP-F of 100 μg/ml.

c) Evidence of MEP-F for Decreasing the HCV Activity in Hepatitis C as well as HIV Activity:

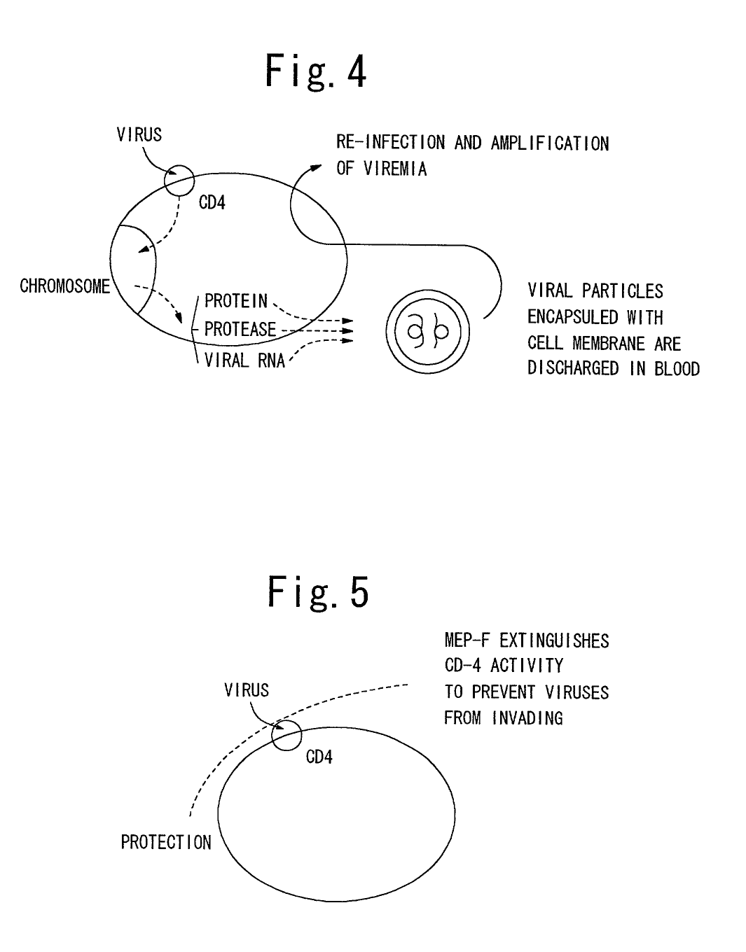

As shown in FIGS. 4 and 5, while viruses invade in the cell by being mediated with CD4 and chemokine receptors as cell side virus receptors, MEP-F extinguishes the virus receptors by cleaving CD4 from the cell membrane depending on the concentration and reaction time. Accordingly, the virus is prevented from invading into the cell. Furthermore, expression of a variety of adhesive factors is enhanced by invasion of viruses. The enzymes are able to act on these cell factors, and performs inhibitory regulation of expression of these factors.

These enzymes are endoproteases in nature, and infectious property of the virus decreases by allowing them to react with the virus. Therefore, it is conjectured that the enzyme also act on the virus' own protein, and breaks the site of the virus required for invading into the cell by the action of the enzyme.

Recently, it is suggested that the metallo-endopeptidase into which MEP-F is classified is related to activation of various lymphocyte, monocyte and macrophage family cells. Therefore, the enzyme is conjectured to probably act on the cells, and primarily and secondarily regulates virus production.

The enzyme according to the present invention having the chemical and physical properties as described above acts on synthetic substrates shown in Table 1A, and comprises the amino acid composition as shown in Table 1B.

TABLE 1A

| | SYNTHETIC |
|---|---|
| ENZYME | SUBSTRATE |
| ENZYME OF THE | Z-Gly-Gly-Leu-pNA |
| PRESENT INVENTION | ↑ ↑ ↑ |
| THERMOLYCINE | |
| ENZYME OF THE | Pyr-Phe-Leu-pNA |
| PRESENT INVENTION | ↑ ↑ ↑ |
| THERMOLYCINE | |

The arrows in the table indicate cleavage sites.

TABLE 1B

| | MOL % | |
|---|---|---|
| AMINO | HYDROLYSIS WITH 4N | HYDROLYSIS WITH 6N HCl |
| ACID | CH₃SO₃ (24 HRS) | (24 HRS) |
| ASP | 13.0 | 13.1 |
| THR | 9.9 | 10.1 |
| SER | 7.5 | 7.8 |
| GLU | 5.0 | 5.0 |
| GLY | 15.2 | 15.8 |
| ALA | 9.8 | 9.7 |
| CYS | 0.36 | 0.27 |
| VAL | 5.7 | 5.7 |
| MET | 0.98 | 0.97 |
| ILE | 2.7 | 2.7 |
| LEU | 5.8 | 5.8 |
| TYR | 6.8 | 6.8 |
| PHE | 2.4 | 2.5 |
| LYS | 3.5 | 3.5 |
| HIS | 2.7 | 2.7 |
| ARG | 2.7 | 2.8 |
| TRP | 1.5 | 0.7 |
| PRO | 4.5 | 4.5 |

### EXAMPLE 1

Therapeutic Efficacy of MEP-F for Treatment of AIDS Patient:

Case Report

Patient: 49 years old male

(i) Chief complaints: pharynx pain and dizziness

(ii) Clinical findings: Nystagmus with a predominance of right direction and deviated walk to the left side were noted in audiometry and equilibrium function test.

(iii) Laboratory findings: In hematology and blood chemistry tests, no abnormal findings were noted in red blood cell counts, white blood cell counts, hematocrit, GOT, GPT, γ-GTP and total cholesterol levels. No abnormal finding was also noted in the urine test.

(iv) Results of tests for viral infections: No symptoms of hepatitis B and hepatitis C and no abnormal findings were noted in serologic tests for HBV, HCV and antigens. Positive responses were noted in the HIV test and ELISA test, and the result of reconfirmation by the western blot technique also positive.

(iv) Progress following treatment: Daily administration of six tablets of MEP-F was initiated. After dosing for eight weeks, treatment was suspended for three weeks. Then, dosing was resumed and maintained for three weeks. This dosing regimen, comprising three weeks suspension and three weeks administration, was the repeated. AT 25 weeks after initiation of the therapy, the tests using ELISA assay and western blot technique revealed that blood sample from the patient was negative for HIV. The dosing regimen was repeated for further 12 weeks and the clinical course of the patient is now being followed up. MEP-F was shown to be effective for the treatment of HIV infection.

### EXAMPLE 2

Therapeutic Efficacy of MEP-F for Treatment of Hepatitis B Patient:

Case Report

Patient: 47 years old male

(i) Diagnosed disease: hepatitis B, chronic hepatitis

(ii) Chief complaints: mild fatigue

(iii) History of present illness: While the patient has developed fatigue approximately a month ago, he had a good appetite. Other abnormal findings including headache were not noted.

(iv) Present conditions: While the patient visited the hospital as he was diagnosed as hepatitis B by a medical check, no abnormal findings was noted on the abdomen.

(v) Results of hepatic examination: The surface of the liver was smooth and no abnormal finding was noted by hepatic palpation.

(vi) Results of liver function test: See Table 2

(vii) Treatment with the drug according to the present invention: Six capsules of the drug was orally administered once a day. After 12 months, clinical tests showed that the HBS and HBE indices for hepatitis B, and the rate of inhibition of HBE antibody turned to be normal. A weaning administration therapy was concomitantly used.

Table 2 shows the results of the hepatic function test, indicating therapeutic efficacy of MEP-F against hepatitis B.

TABLE 2
Therapeutic Efficacy of MEP-F Against Hepatitis B:
Result of Hepatic Function Test

| | | BEFORE | | | | | | | | | | | | |
|---|---|---|---|---|---|---|---|---|---|---|---|---|---|---|
| TEST | NORMAL | ADMINIS | PROGRESS OF THERAPEUTIC EFFICACY (MONTHS) | | | | | | | | | | | |
| ITEM | LEVEL | TRATION | 1 | 2 | 3 | 4 | 5 | 6 | 7 | 8 | 9 | 10 | 11 | 12 |
| α2 MG | 118-240 | 118-240 | 227 | 210 | 254 | 227 | 211 | 228 | 229 | 233 | 247 | 270 | 257 | 263 |
| HBS | LT. 0.9 | 63.8 | 45.6 | 79.8 | 48.1 | 40.7 | 37.8 | 48.4 | 24.6 | 14.7 | 17.6 | 5.9 | 2.3 | 1.0 |
| ANTI- | | | | | | | | | | | | | | |
| GEN | | | | | | | | | | | | | | |
| INDEX | | | | | | | | | | | | | | |
| HEB | LT. 0.9 | 0.3 | 0.3 | 0.2 | 0.2 | 0.3 | 0.2 | 0.2 | 0.2 | 0.3 | 0.3 | 0.2 | 0.2 | 0.2 |
| ANTI- | | | | | | | | | | | | | | |
| GEN | | | | | | | | | | | | | | |
| INDEX | | | | | | | | | | | | | | |
| RATE OF | LT. 29.9 | 99.1 | 99.5 | 99.7 | 99 | 99.8 | 99.7 | 98.8 | 99.4 | 98.7 | 88.8 | 82.6 | 82.5 | 78.3 |
| INHIBI- | | | | | | | | | | | | | | |
| TION | | | | | | | | | | | | | | |
| OF HBE | | | | | | | | | | | | | | |
| ANTI- | | | | | | | | | | | | | | |
| GEN | | | | | | | | | | | | | | |
| TOTAL | 0.2-1.0 | 0.5 | 0.5 | 0.8 | 0.7 | 0.9 | 0.8 | 0.7 | 0.8 | 0.7 | 0.9 | 0.7 | 0.8 | 0.9 |
| BILI- | | | | | | | | | | | | | | |
| RUBIN | | | | | | | | | | | | | | |
| DIRECT | 0-0.4 | 0.4 | 0.3 | 0.4 | 0.3 | 0.3 | 0.2 | 0.3 | 0.2 | 0.4 | 0.3 | 0.3 | 0.4 | 0.3 |
| BILI- | | | | | | | | | | | | | | |
| RUBIN | | | | | | | | | | | | | | |
| TTT | 0.5-6.5 | 1.1 | 3.9 | 0.3 | 0.8 | 1.6 | 1.5 | 1.1 | 0.8 | 0.8 | 0.9 | 0.8 | 1.5 | 0.7 |
| ZTT | 2.3-12.0 | 2.9 | 3.7 | 3.7 | 3.5 | 2.9 | 3.3 | 4.3 | 4.9 | 4.6 | 4.1 | 5.1 | 4.6 | 4.1 |
| GOT | 10-40 | 114 | 85 | 78 | 80 | 65 | 62 | 66 | 52 | 42 | 45 | 41 | 40 | 38 |
| GPT | 5-45 | 129 | 7 | 90 | 86 | 67 | 63 | 67 | 58 | 47 | 45 | 46 | 45 | 44 |
| AL-P | 2.0-10.0 | 21.1 | 18.3 | 13.5 | 10.6 | 10.1 | 9.7 | 9.8 | 8 | 7.8 | 8.3 | 8.5 | 7.6 | 7.5 |
| LDH | 220-480 | 437 | 371 | 371 | 427 | 367 | 350 | 614 | 354 | 405 | 398 | 347 | 357 | 370 |
| LAP | 25-50 | 27 | 26 | 33 | 30 | 30 | 30 | 29 | 31 | 29 | 28 | 28 | 26 | 29 |
| γ-GTP | BELOW | 14 | 13 | 11 | 12 | 13 | 10 | 11 | 11 | 13 | 12 | 11 | 10 | 14 |
| | 60 | | | | | | | | | | | | | |
| TOTAL | 150-219 | 192 | 195 | 198 | 212 | 194 | 156 | 170 | 182 | 202 | 198 | 186 | 171 | 165 |
| CHOLE- | | | | | | | | | | | | | | |
| STEROL | | | | | | | | | | | | | | |
| TOTAL | 0.5-8.2 | 7.0 | 6.6 | 7.1 | 7.2 | 7.5 | 7.5 | 7.1 | 6.8 | 7.1 | 7.3 | 7 | 6.8 | 7.5 |
| PROTEIN | | | | | | | | | | | | | | |
| ALBU- | 56-68 | 58.4 | 55.3 | 59.4 | 58.4 | 60 | 59.7 | 56.6 | 57.7 | 59.2 | 60.1 | 59.8 | 56.6 | 55.8 |
| MIN | | | | | | | | | | | | | | |
| A/G | 1.3-2.0 | 1.63 | 1.62 | 1.73 | 1.61 | 1.50 | 1.43 | 1.34 | 1.60 | 1.47 | 1.54 | 1.50 | 1.29 | 1.28 |

### EXAMPLE 3

Therapeutic Efficacy of MEP-F for Treatment of Hepatitis C Patient

Case Report

Patient: 68 years old female

(i) Diagnosed disease: hepatitis C, chronic hepatitis

(ii) Chief complaints: generalized fatigue and loss of appetite

(iii) History of present illness: The patient complains generalized fatigue and loss of appetite since about seven month ago together with lose of vigor to work.

(iv) Present conditions: The patient had facial pallor which was tinged with yellow. Although a slightly swollen abdomen was noticed, no other abnormal findings such as retention of ascites fluid were noted.

(v) Echo test and X-ray test: No abnormal findings were found in both livers and spleen. The gall bladder was slightly swollen. No gallstone was found. No abnormal findings were noted in the pancreas, large and small intestines, stomach and duodenum.

(vi) Liver: The surface of the liver was smooth. Although the hepatic duct was constricted mildly, no atrophy and local abnormality were noted in the hepatic tissue. The results of the hepatic function test are shown in Table 3.

Table 3 shows the results of the hepatic function tests, indicating therapeutic efficacy of MEP-F.

TABLE 3
Therapeutic Efficacy of MEP-F Against Hepatitis C:
Result of Hepatic Function Test

| | | BEFORE | | | | | | | | | | | | |
|---|---|---|---|---|---|---|---|---|---|---|---|---|---|---|
| TEST | NORMAL | ADMINIS | PROGRESS OF THERAPEUTIC EFFICACY (MONTHS) | | | | | | | | | | | |
| ITEM | LEVEL | TRATION | 1 | 2 | 3 | 4 | 5 | 6 | 7 | 8 | 9 | 10 | 11 | 12 |
| α2 MG | 118-240 | 270 | 243 | 255 | 250 | 247 | 250 | 260 | 247 | 275 | 285 | 260 | 278 | 285 |
| HCV | LT. 0.15 | 4.74 | 4.63 | 13.6 | 5.30 | 14.3 | 4.21 | 2.4 | 4.32 | 1.12 | 1.23 | 0.25 | 0.16 | 0.15 |
| ANTI- | | | | | | | | | | | | | | |
| GEN | | | | | | | | | | | | | | |
| INDEX | | | | | | | | | | | | | | |
| HCV | LT. 1.0 | 31.6 | 30.9 | 90.7 | 35.3 | 95.3 | 26.4 | 9.6 | 9.8 | 2.7 | 2.8 | 1.6 | 1.2 | 1 |
| ANTI- | | | | | | | | | | | | | | |
| GEN | | | | | | | | | | | | | | |
| UNIT | | | | | | | | | | | | | | |
| QUAN- | RNA 0 | 10 | 9 | 9 | 10 | 7 | 6 | 4 | 7 | 3 | 4 | 3 | 1 | 0 |
| TIFI- | | | | | | | | | | | | | | |
| CATION | | | | | | | | | | | | | | |
| OF MCV | | | | | | | | | | | | | | |
| ANTI- | | | | | | | | | | | | | | |
| GEN BY | | | | | | | | | | | | | | |
| PCR | | | | | | | | | | | | | | |
| TOTAL | 0.2-1.0 | 0.7 | 0.5 | 0.4 | 0.6 | 0.4 | 0.5 | 0.5 | 0.4 | 0.4 | 0.5 | 0.5 | 0.4 | 0.5 |
| BILI- | | | | | | | | | | | | | | |
| RUBIN | | | | | | | | | | | | | | |
| DIRECT | 0-0.4 | 0.4 | 0.2 | 0.3 | 0.4 | 0.2 | 0.3 | 0.2 | 0.1 | 0.1 | 0.2 | 0.1 | 0.2 | 0.2 |
| BILI- | | | | | | | | | | | | | | |
| RUBIN | | | | | | | | | | | | | | |
| TTT | 0.5-6.5 | 17.4 | 14.3 | 13.7 | 13.8 | 11.9 | 9.6 | 8.4 | 8.7 | 7.3 | 6.7 | 6.5 | 6.6 | 6.5 |
| ZTT | 2.3-12.0 | 28.3 | 27.1 | 23 | 23.9 | 19.5 | 16.8 | 13.6 | 15.7 | 12.2 | 12 | 11.8 | 11.7 | 11.6 |
| GOT | 10-40 | 173 | 146 | 152 | 174 | 126 | 96 | 74 | 78 | 62 | 57 | 55 | 56 | 51 |
| GPT | 5-45 | 172 | 145 | 150 | 168 | 128 | 101 | 83 | 76 | 61 | 55 | 47 | 47 | 46 |
| AL-P | 2.0-10.0 | 23.4 | 21.1 | 20.8 | 21.1 | 18.5 | 14.7 | 13.9 | 12.4 | 11.6 | 10.9 | 9.9 | 9.8 | 9.7 |
| LDH | 220-480 | 485 | 396 | 414 | 388 | 342 | 379 | 404 | 382 | 398 | 360 | 375 | 381 | 342 |
| LAP | 25-50 | 50 | 39 | 42 | 43 | 44 | 43 | 44 | 42 | 39 | 38 | 41 | 42 | 39 |
| γ-GTP | BELOW | 28 | 29 | 39 | 44 | 43 | 42 | 42 | 43 | 44 | 43 | 42 | 43 | 42 |
| | 60 | | | | | | | | | | | | | |
| TOTAL | 150-219 | 132 | 127 | 118 | 126 | 132 | 125 | 135 | 127 | 115 | 123 | 117 | 109 | 115 |
| CHOLE- | | | | | | | | | | | | | | |
| STEROL | | | | | | | | | | | | | | |
| TOTAL | 0.5-8.2 | 7.2 | 7.4 | 7.3 | 7.0 | 6.9 | 7.1 | 6.6 | 7.1 | 7.2 | 7.4 | 7.0 | 7.3 | 7.5 |
| PROTEIN | | | | | | | | | | | | | | |
| ALBU- | 56-68 | 41.3 | 41.6 | 40.3 | 40.5 | 41.8 | 40.6 | 39.7 | 41.8 | 41.8 | 41.9 | 40.1 | 45.4 | 51.3 |
| MIN | | | | | | | | | | | | | | |
| A/G | 1.3-2.0 | 0.76 | 0.76 | 0.71 | 0.74 | 0.79 | 0.73 | 0.71 | 0.72 | 0.74 | 0.73 | 0.71 | 0.79 | 0.81 |

### STATISTICS

In addition to the clinical cases above, statistics were compiled with respect to other cases on the hepatitis B and hepatitis C, and the following results were obtained.

Table 4 shows the progress of therapy of six cases of hepatitis B, showing therapeutic efficacy of MEP-F on hepatitis B.

Table 5 shows the progress of therapy of six cases of hepatitis C, showing therapeutic efficacy of MEP-F on hepatitis C.

TABLE 4
Progress of Therapy by MEP-F in 6 cases of Hepatitis B

| MEAN VALUE OF SIX | BEFORE | | | | | | | | | | | | |
|---|---|---|---|---|---|---|---|---|---|---|---|---|---|
| CASES OF | ADMINIS | PROGRESS OF THERAPEUTIC EFFICACY (MONTHS) | | | | | | | | | | | |
| HEPATITIS B | TRATION | 1 | 2 | 3 | 4 | 5 | 6 | 7 | 8 | 9 | 10 | 11 | 12 |
| HBS ANTIGEN INDEX | 71.4 | 57.4 | 47.7 | 28 | 25.4 | 14.9 | 9.45 | 4.45 | 1.30 | 2.00 | 1.23 | 1.00 | 1.11 |
| (LT. 0.9) | | | | | | | | | | | | | |
| HBE ANTIGEN INDEX | 0.3 | 0.3 | 0.2 | 0.2 | 0.3 | 0.2 | 0.2 | 0.3 | 0.2 | 0.2 | 0.3 | 0.2 | 0.3 |
| (LT. 0.9) | | | | | | | | | | | | | |
| RATE OF | 99.1 | 99.5 | 99.7 | 99.8 | 99.8 | 99.7 | 98.8 | 98.7 | 98.7 | 88.8 | 82.6 | 82.5 | 78.3 |
| INHIBITION OF HB | | | | | | | | | | | | | |
| ANTIGEN | | | | | | | | | | | | | |
| (LT. 0.9) | | | | | | | | | | | | | |

TABLE 5
Progress of Therapy by MEP-F in 6 cases of Hepatitis C

| MEAN VALUE OF SIX | BEFORE | | | | | | | | | | | | |
|---|---|---|---|---|---|---|---|---|---|---|---|---|---|
| CASES OF | ADMINIS | PROGRESS OF THERAPEUTIC EFFICACY (MONTHS) | | | | | | | | | | | |
| HEPATITIS B | TRATION | 1 | 2 | 3 | 4 | 5 | 6 | 7 | 8 | 9 | 10 | 11 | 12 |
| HCV ANTIGEN INDEX | 2.65 | 2.36 | 1.99 | 4.26 | 2.10 | 1.46 | 0.87 | 0.84 | 9.30 | 0.33 | 0.21 | 0.17 | 0.17 |
| (LT. 0.15) | | | | | | | | | | | | | |
| HCV ANTIGEN UNIT | 31.6 | 30.9 | 51.9 | 35.3 | 37.8 | 26.4 | 9.6 | 9.8 | 2.7 | 2.8 | 1.6 | 1.2 | 1.0 |
| (LT. 1.0) | | | | | | | | | | | | | |
| QUANTIFICATION | 0 | 0 | 0 | 0 | 1 | 1 | 2 | 2 | 2 | 3 | 4 | 5 | 5 |
| OF HCV ANTIGEN BY | | | | | | | | | | | | | |
| PCR | | | | | | | | | | | | | |
| (NO OF EXAMPLES | | | | | | | | | | | | | |
| OF 10° | | | | | | | | | | | | | |

Viruses should pass through CD4 receptors on the surface of the cell membrane for invading into the cell. However, since MEP-F extinguishes the activity of CD4 to prevent the viruses from invading into the cell, growth and proliferation of the virus are inhibited, manifesting preventive and therapeutic efficacy for viral infection.

What is claimed is:

1. A preventive and therapeutic antiviral drug for AIDS, hepatitis B, hepatitis C and influenza containing MEP-F (metalloendopeptidase-F) and other proteases as effective ingredients.

2. A preventive and therapeutic antiviral drug according to claim 1 having the chemical and physical properties comprising:
a molecular weight of 43,000±5,000 as determined by SDS electrophoresis, and a molecular weight of 28,000±7,000 as determined by gel filtration using TSK 3000 SW;
an isoelectric point pI of 4.76±1.0;
substrate specificity against casein, -X-Leu-Y- or Cbz-Cly-Leu-NH₂, and X-Phe-Y- or Cbz-Gly-Phe-NH₂ for cleaving at X-Phe or Gly-Lue bond, and at X-Phe or Gly-Phe bond;
an optimum pH range of 5.5 to 9.5, or 6 to 8, for enzyme activity;
a stable pH range of 4 to 10, or 6 to 8, for enzyme activity;
inhibitors such as EDTA, phosphoramidone and leucine
solubility in water and insolubility in acetone and ethanol; and
a Rf value of 0.08 in 7.0% polyacrylamide gel electrophoresis.

3. A preventive and therapeutic antiviral drug according to claim 1 or 2 containing either one or at least two or more of the proteases selected from trypsin, α-chymotrypsin, bromelain, papain, Serratia protease, peptidase, sepahrase, pronase, prozyme, urokinase, pancreatin, fibrinolysin, elecodase, collagenase, gelatinase and matrix metalloprotease.
